(19) United States
(12) Patent Application Publication
(10) Pub. No.: US 2010/0262233 A1
(43) Pub. Date: Oct. 14, 2010

(54) Mitral Valve Coaptation Plate For Mitral Valve Regurgitation

(75) Inventor: Zhaoming He, (US)

Correspondence Address: Roman Aguilera III;TTUS Office of Technology Commercialization, Box 42007, Lubbock, TX, 79409-2007, US

(73) Assignee: Texas Tech University System

(21) Appl. No.: 12/422,287

(22) Filed: Apr. 12, 2009

(51) Int. Cl. A61F 2/24

(52) U.S. Cl. 623/2.36; 623/2.41

(57) ABSTRACT

A method and apparatus directed to the repair of regurgitant mitral valves. Mitral valve regurgitation occurs due to miscoaptation of mitral valve leaflets. The mitral valve repair apparatus of the present invention is comprised of a tongue plate which is supported by a suture ring. The apparatus is inserted into the mitral valve orifice with the suture ring sutured to the mitral valve annulus placing the tongue plate between the two mitral valve leaflets. When the mitral valve opens, blood flows through the orifices of the apparatus. When the mitral valve closes, the two miscoaptated mitral valve leaflets cover the orifices on the apparatus and the tongue plate blocks the hole formed by leaflets and seals the leaky flow.

### CROSS-REFERENCE TO RELATED APPLICATIONS

Not applicable

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates to the repair of a heart's mitral valve. More specifically, the present invention relates to an apparatus and method for repairing regurgitating mitral valves.

### BACKGROUND OF THE INVENTION

Without limiting the scope of the disclosed apparatus and method, the background is described in connection with a novel approach to the repair of a regurgitating mitral valve.

The human heart consists of four chambers: the left atrium, the left ventricle, the right atrium and the right ventricle. The atria are isolated from their respective ventricles by one-way valves located at the respective atrial-ventricular junctions. These valves are identified as the mitral (or bicuspid) valve on the left side of the heart, and tricuspid valve on the right side of the heart. The exit valves from the left and right ventricles are identified as the aortic and pulmonary valves, respectively.

In normal operation, the leaflets of the mitral valve open as the left ventricle dilates thereby permitting blood to flow from the left atrium into the left ventricle. The leaflets then close during the contraction cycle of the left ventricle, thereby preventing the blood from returning to the left atrium and forcing the blood to exit the left ventricle through the aortic valve.

When a mitral valve functions properly, it prevents regurgitation of blood from the ventricle into the atrium when the ventricle contracts. In order to withstand the substantial backpressure and prevent regurgitation of blood into the atrium during the ventricular contraction, the cusps are held in place by fibrous cords that anchor the valve cusps to the muscular wall of the heart.

The mitral valve is a complex load bearing structure that consists of an annulus, two leaflets, chordae, papillary muscles, and the underlying left ventricular myocardium. The anterior and posterior leaflets are attached to the annulus. The annulus is an anatomical structure joining the leaflets and the left ventricle wall. It is divided into the fibrous annulus in the anteromedial section and the myocardium annulus in the posterolateral section, according to annulus histology. The chordae originates from the papillary muscles and attach to the leaflets. They prevent the leaflets from prolapsing into the left atrium during systole. The papillary muscles are attached to the wall of the left ventricle.

A mitral valve may become defective or damaged, resulting in a regurgitant mitral valve. This is where the mitral valve leaflets do not close properly and blood flows backward from the left ventricle to the left atrium during systole. In addition, pathological alterations affecting any of the mitral valve's structures, such as annulus dilatation, papillary muscle displacement, leaflet calcification, and chordae rupture or elongation can lead to altered valve function and also cause mitral valve regurgitation. Mitral valve regurgitation can cause pulmonary congestion and a dilated left ventricle which can ultimately result in heart failure.

Mitral valve replacement and repair can be conducted to correct mitral valve regurgitation. Mitral valve repair is now a preferable surgical approach to whole valve replacement because of fewer traumas and less complication. Common mitral valve repair techniques include triangular or quadrangular resection, slide annuloplasty, ring annuloplasty, chordal cutting and transposition, artificial chord use and, recently, percutaneous technologies. The present invention proposes a novel mitral valve repair device, a mitral valve coaptation plate.

### BRIEF SUMMARY OF THE INVENTION

The present invention, therefore, provides a method and apparatus to repair a regurgitant mitral valve using an apparatus to support the coaptation of the mitral valve leaflets. The apparatus is comprised of a tongue plate supported by a suture ring and is implanted into the patient's heart by various means such as open heart surgery or other percutaneous methods. It is the first apparatus to repair a regurgitant mitral valve in such a manner and is distinct from existing heart valve repair methods and devices, such as annuloplasty rings, mitral valve plugs, and mitral valve webs. In addition, these other repair methods are not as effective against mitral valve regurgitation due to ischemic disease. For instance, with an annuloplasty ring, there is a fifty percent chance of mitral regurgitation reoccurring in six to seven years. Therefore, a new device and approach is needed in the art.

In summary, the present invention discloses an improved method and apparatus for repairing a regurgitant mitral valve. More specifically, by extension, the disclosed method and apparatus can be used to repair miscoaptation in the tricuspid valve.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a depiction of the human heart and heart valves;

FIG. 2 is a depiction of the mitral valve structure;

FIG. 3A is a depiction of the lateral view of the mitral valve in the closed position;

FIG. 3B is a depiction of the atrial view of the mitral valve in the closed position;

FIG. 4A is a depiction of the lateral view of mitral valve in the closed position showing miscoaptation and leakage during systole;

FIG. 4B is a depiction of the of the atrial view of the mitral valve in the closed position showing miscoaptation and leakage during systole;

FIG. 5A is a depiction of the lateral view of the mitral valve coaptation plate in accordance with embodiments of the disclosure;

FIG. 5B is a depiction of the axis view of the mitral valve coaptation plate in accordance with embodiments of the disclosure;

FIG. 5C is a depiction of the apical view of the mitral valve coaptation plate in accordance with embodiments of the disclosure;

FIG. 5D is a depiction of the anterior view of the mitral valve coaptation plate in accordance with embodiments of the disclosure;

FIG. 5E is a depiction of the lateral cross-section view of the mitral valve coaptation plate in accordance with embodiments of the disclosure;

FIG. 5F is a depiction of the apical cross-section view of the mitral valve coaptation plate in accordance with embodiments of the disclosure;

FIG. 6A is a depiction of the lateral view of the mitral valve coaptation plate in the mitral valve when the leaflets are closed in accordance with embodiments of the disclosure;

FIG. 6B is a depiction of the atrial view of the mitral valve coaptation plate in the mitral valve when the leaflets are closed in accordance with embodiments of the disclosure;

FIG. 7A is a depiction of the lateral view of the mitral valve coaptation plate in the mitral valve when the leaflets are open in accordance with embodiments of the disclosure;

FIG. 7B is a depiction of the atrial view of the mitral valve coaptation plate in the mitral valve when the leaflets are open in accordance with embodiments of the disclosure;

FIG. 8 is a depiction of the apical view of the mitral valve coaptation plate in the mitral valve when the leaflets are closed in accordance with embodiments of the disclosure;

FIG. 9 is a depiction of the apical view of the mitral valve coaptation plate in the mitral valve when the leaflets are open in accordance with embodiments of the disclosure;

FIG. 10A is a depiction of the lateral view of the mitral valve coaptation plate with extension rods for chordal repair when the leaflets are closed in accordance with embodiments of the disclosure;

FIG. 10B is a depiction of the atrial view of the mitral valve coaptation plate with extension rods for chordal repair when the leaflets are closed in accordance with embodiments of the disclosure;

FIG. 10C is a depiction of the lateral view of the mitral valve coaptation plate with extension rods for chordal repair in accordance with embodiments of the disclosure;

FIG. 10D is a depiction of the apical view of the mitral valve coaptation plate with extension rods for chordal repair in accordance with embodiments of the disclosure;

FIG. 11A is a depiction of the lateral view of the mitral valve coaptation plate with an extension bar with holes for chordal repair when the leaflets are closed in accordance with embodiments of the disclosure;

FIG. 11B is a depiction of the atrial view of the mitral valve coaptation plate with an extension bar with holes for chordal repair when the leaflets are closed in accordance with embodiments of the disclosure;

FIG. 11C is a depiction of the lateral view of the mitral valve coaptation plate with an extension bar with holes for chordal repair in accordance with embodiments of the disclosure;

FIG. 11D is a depiction of the apical view of the mitral valve coaptation plate with an extension bar with holes for chordal repair in accordance with embodiments of the disclosure;

FIG. 12A is a depiction of the tongue plate at an angle θ to the annulus plane in accordance with embodiments of the disclosure;

FIG. 12B is a depiction of the tongue plate with a wedge shape at an angle θ to the annulus plane in accordance with embodiments of the disclosure;

FIG. 12C is a depiction of the tongue plate with a curved wedge shape at an angle θ to the annulus plane in accordance with embodiments of the disclosure.

### DETAILED DESCRIPTION OF THE INVENTION

Disclosed herein is an improved method and apparatus for repairing a heart's regurgitating mitral valve. The numerous innovative teachings of the present invention will be described with particular reference to several embodiments (by way of example, and not of limitation).

All figures referred to will use the following descriptions for reference numbers: left atrium (1), mitral valve (2), annulus (2.1), anterior leaflet (2.2), posterior leaflet (2.3), chordate (2.4), papillary muscle (3), mitral valve coaptation plate (4), suture ring (4.1), tongue plate (4.2), extension rods or bar (4.3), left ventricle (5), aortic valve (6), aortic artery (7).

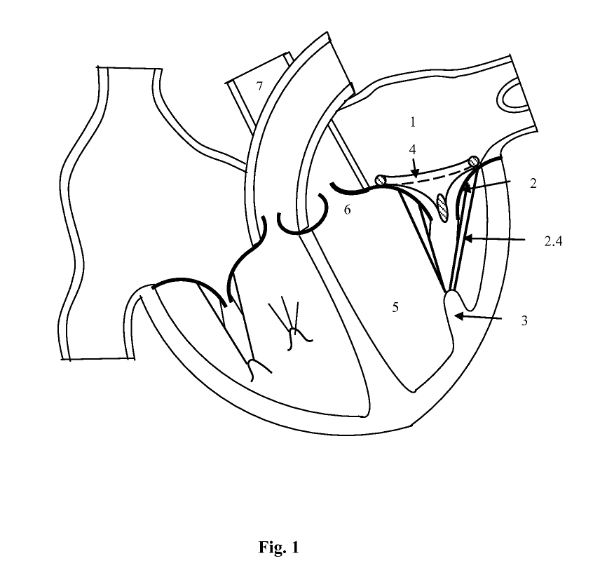

Reference is first made to FIG. 1, a schematic of a human heart and its four heart valves. In the left heart, the mitral valve (2) lies between the left atrium (1) and the left ventricle (5) to control blood flow from the left atrium (1) to the left ventricle (5). A dysfunctional mitral valve causes regurgitation where blood flows backward from the left ventricle (5) to the left atrium (1) during systole. Mitral regurgitation can cause pulmonary congestion and a dilated left ventricle which ultimately can cause heart failure and mortalities. FIG. 1 also shows the mitral valve coaptation plate (4) implanted in a regurgitant mitral valve (2) in the left heart. Orientation of the coaptation plate is demonstrated from this depiction.

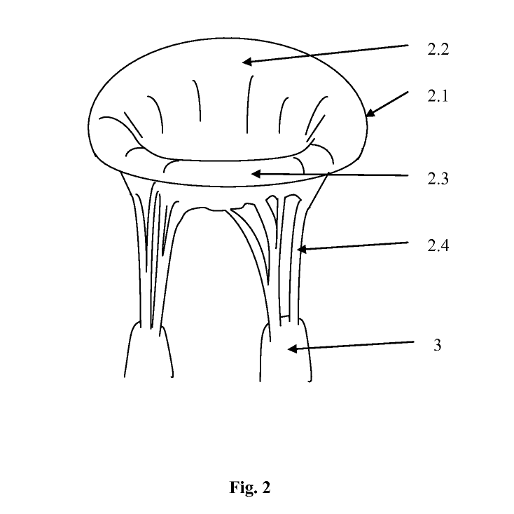

Reference is now made to FIG. 2, wherein a mitral valve schematic is presented. The mitral valve is a complex load bearing structure that consists of an annulus (2.1), two leaflets (2.2 and 2.3), chordae (2.4), papillary muscles (3), and the underlying left ventricular myocardium. The anterior (2.2) and posterior leaflets (2.3) are attached to the annulus (2.1). The annulus (2.1) is an anatomical structure joining the leaflets (2.2 and 2.3) and left ventricle (5) wall. It is divided into the fibrous annulus in the anteromedial section and the myocardium annulus in the posterolateral section, according to annulus histology. The chordae (2.4) originate from papillary muscles (3) and attach to the leaflets (2.2 and 2.3). They prevent leaflets (2.2 and 2.3) from prolapsing into the left atrium (1) during systole. The papillary muscles (3) are attached to the wall of the left ventricle.

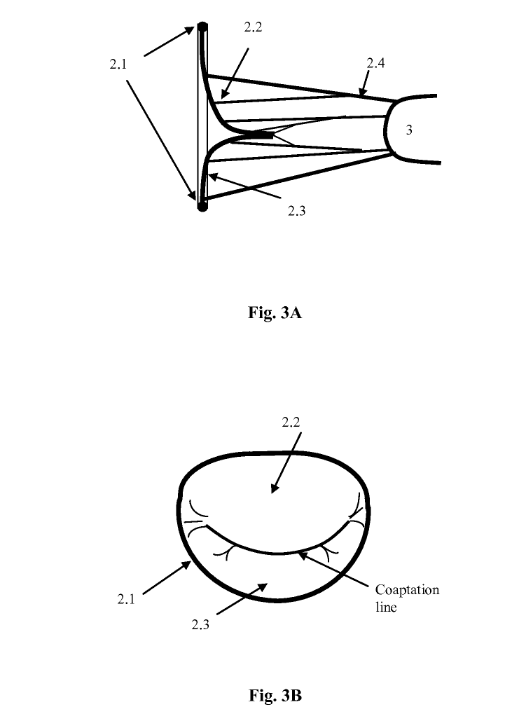

Reference is now made to FIGS. 3A and 3B, wherein a normal functioning mitral valve is shown in the closed position from the lateral and atrial view. In a normal functioning mitral valve, no gaps are present between the leaflets in the closed position. Thus, in a normal functioning mitral valve, regurgitation is not present.

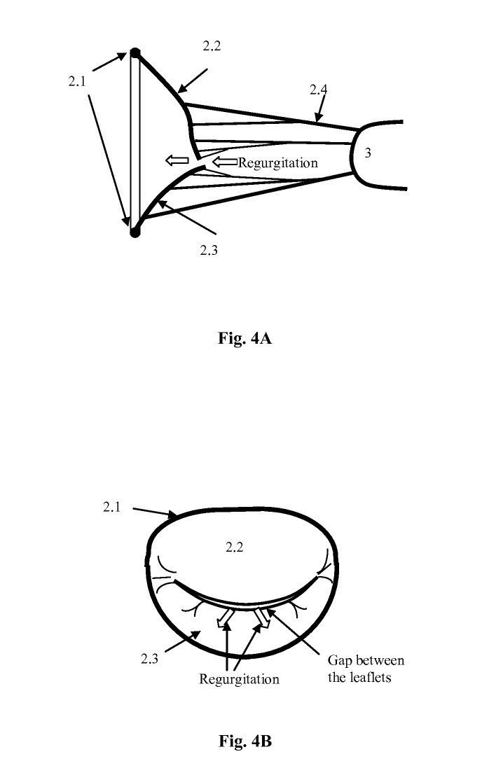

Reference is now made to FIGS. 4A and 4B, wherein a regurgitating mitral valve is shown from the lateral and atrial view. In a regurgitating mitral valve, gaps are present between the leaflets in the closed position. These gaps or miscoaptation of the leaflets allow blood to flow back into the left atrium from the left ventricle during systole. This backflow is referred to as regurgitation.

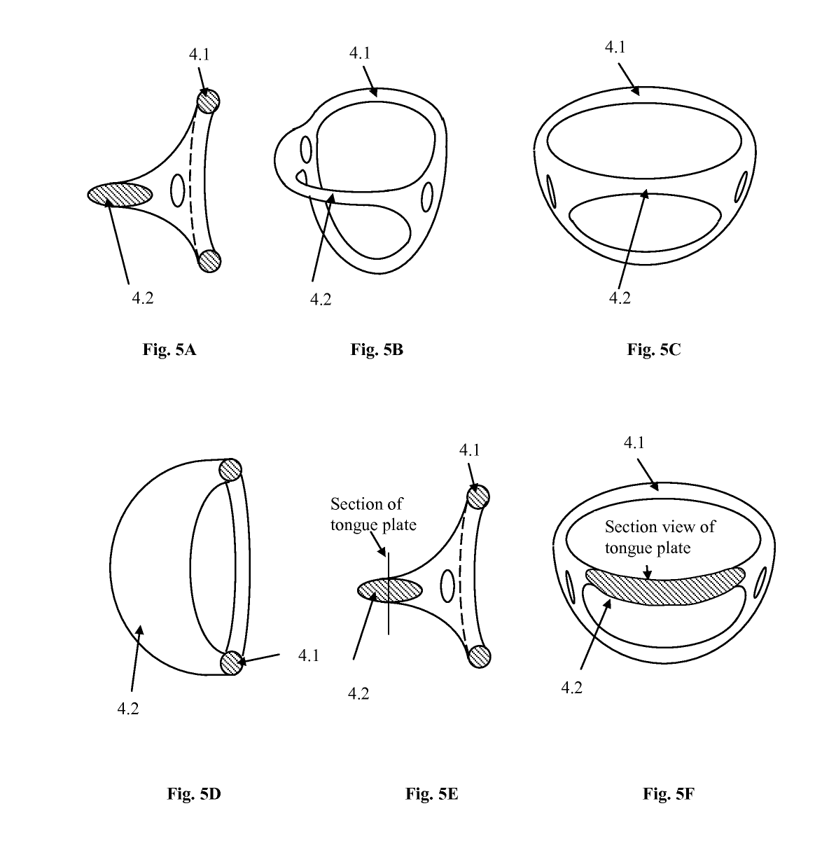

Reference is now made to FIGS. 5A-5F, wherein several embodiment of the mitral valve coaptation plate are shown. The mitral valve coaptation plate is composed of a suture ring (4.1) and a tongue plate (4.2) even if both may be made into a whole body. The suture ring (4.1) allows the apparatus to be attached to the mitral valve annulus (2.1). The suture ring (4.1) looks like a “D” in a saddle shape and matches the size and shape of the native mitral valve annulus (2.1). The anterior section of the suture ring (4.1) is relatively straight from trigone to trigone. The posterior section of the suture ring (4.1) is a half circle. Septal-lateral diameter of the suture ring is controlled so as to be close to the native mitral valve annulus in size and smaller than the dilatated annulus. The suture ring can be a partial ring with separate anterior and posterior sections of the suture ring in which support is needed to connect the partial suture ring and tongue plate. The whole suture ring (4.1) is preferably manufactured from a rigid or semi-rigid material and covered with Dacron material or other coating materials which are compatible to blood. The covering has a soft texture which is suitable for suturing to the mitral valve annulus. The whole suture ring (4.1) can be deformable to match the patient's annulus (2.1) geometry.

The tongue plate (4.2) of the mitral valve coaptation plate (4) is the most important part of the invention. The tongue plate sticks out from the commissural positions of the suture ring (4.1) into the left ventricle (5). The tongue plate (4.2) matches the free edges of the leaflets (2.2 and 2.3) during mitral valve closure, supporting the leaflets and sealing the gap between the leaflets (2.2 and 2.3), thereby preventing mitral valve regurgitation. The plate shape, size and orientation are designed to be forgiving to leaflet positions and sizes in a range depending upon the mechanism of mitral regurgitation. The tongue plate has an angle relative to the annulus plane, depending upon papillary muscle tethering in ischemic mitral regurgitation. The whole tongue plate is semi-rigid, pitching a little between two leaflets, self-adjustable to some extent. The tongue plate (4.2) is made of rigid or semi-rigid materials with adequate strength. The surface is covered by plastic materials such as polyurethane or rigid materials such as pyrolytical carbon or nitinnol. If it is made of plastic materials such as polyurethane, the tongue plate can be deformable by injecting a polymer material which is solidified at the end of repair. The holes in the lateral sides of the tongue plate (4.2) depend upon the commissural leaflet size and shape. Very small holes or no holes are made in the tongue plate (4.2) for small or bad commissural leaflets. These discretionary holes in the tongue plate provide additional blood flow paths during the open mitral valve position which help wash, via blood circulation, additional areas in the mitral valve. FIG. 5D demonstrates how the tongue plate (4.2) may take on various dimensions. The length of the tongue plate, the dimension extending from the plane of the suture ring to the tongue plate's point furthest away in the direction of the left ventricle, can vary.

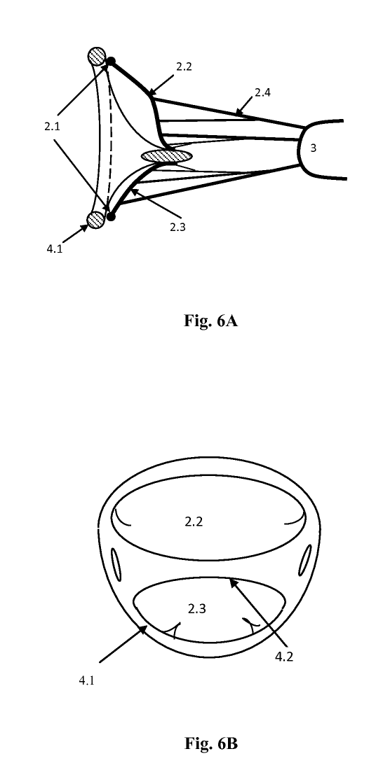

Reference is now made to FIGS. 6A and 6B, wherein a mitral valve coaptation plate is shown in a regurgitating mitral valve from the lateral and atrial view. Both FIGS. 6A and 6B show the mitral valve in the closed position. As can be seen from these illustrations, the mitral valve leaflets cover the openings of the mitral valve coaptation plate. Without the mitral valve coaptation plate, the leaflets would not seal properly. Now, with the mitral valve coaptation plate implanted, the gaps between the leaflets are now filled by the tongue plate of the apparatus.

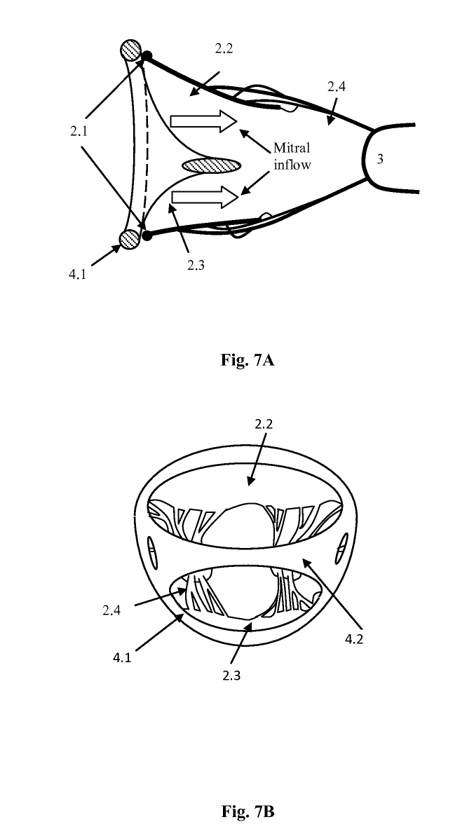

Reference is now made to FIGS. 7A and 7B, wherein a mitral valve coaptation plate is shown in a regurgitating mitral valve from the lateral and atrial view. Both FIGS. 7A and 7B show the mitral valve in the open position. These illustrations show how the blood is allowed to pass through the apparatus in the open position for the mitral valve. The orifices in the apparatus allow the blood to flow through freely.

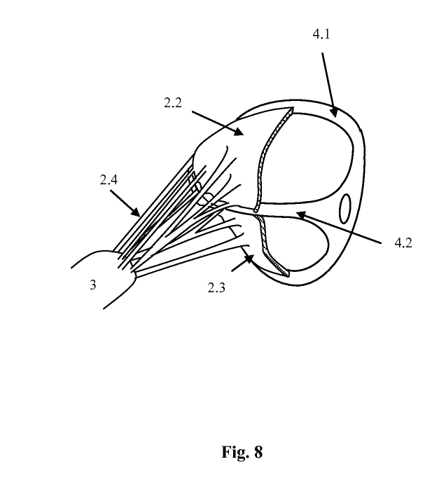

Reference is now made to FIG. 8, wherein the mitral valve coaptation plate is shown in the mitral valve from the apical view and highlights how the leaflets are positioned on the tongue plate when the leaflets are in the closed position.

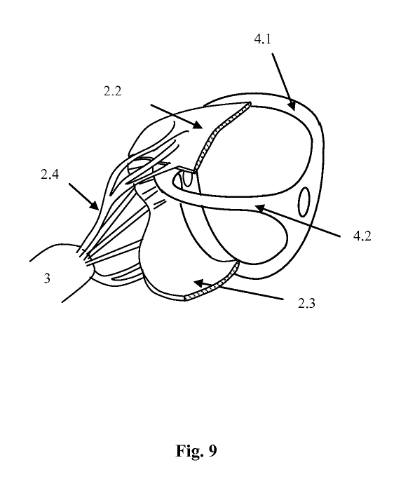

Reference is now made to FIG. 9, wherein the mitral valve coaptation plate is shown in the mitral valve from the apical view and highlights how the leaflets are positioned on the coaptation plate when the leaflets are in the open position.

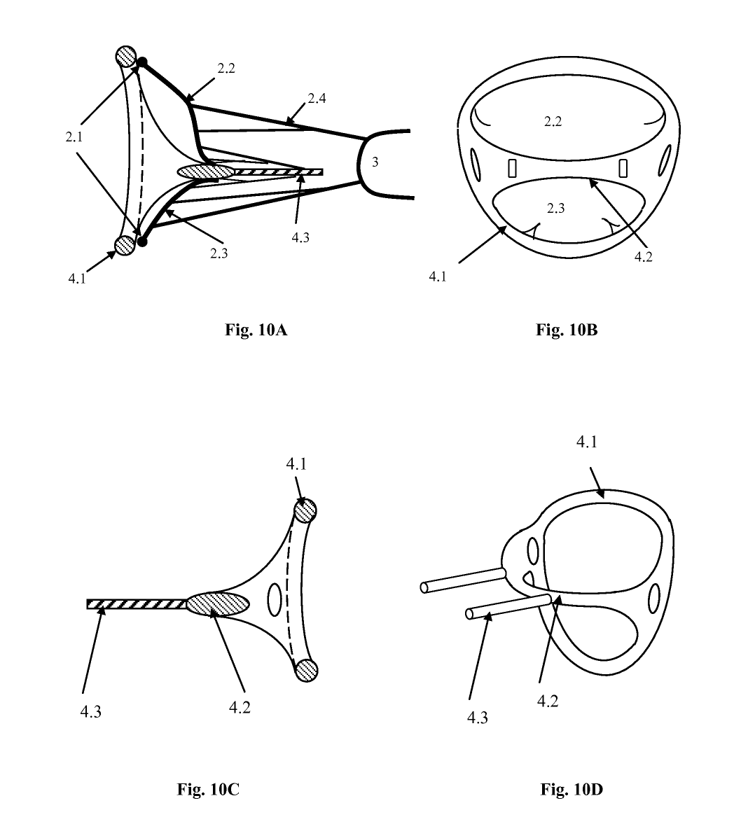

Reference is now made to FIGS. 10A-10D, wherein the coaptation plate is shown with extension rods to help facilitate the attachment of chordae for translocation. The tip of the tongue plate (4.2) can be extended with two rods with hooks which can be used to anchor artificial chordae (polytetrafluoroethylene) or other chordae repair. FIG. 10D shows the rods in the mitral valve coaptation plate. The holes in the lateral sides of the tongue plate (4.2) depend upon the commissural leaflet size and shape. Extra supporting rods can be made between the tongue plate and the anterior or posterior annulus (2.1). The tongue plate can be a complete plate across two commissures or a partial plate with one commissure side.

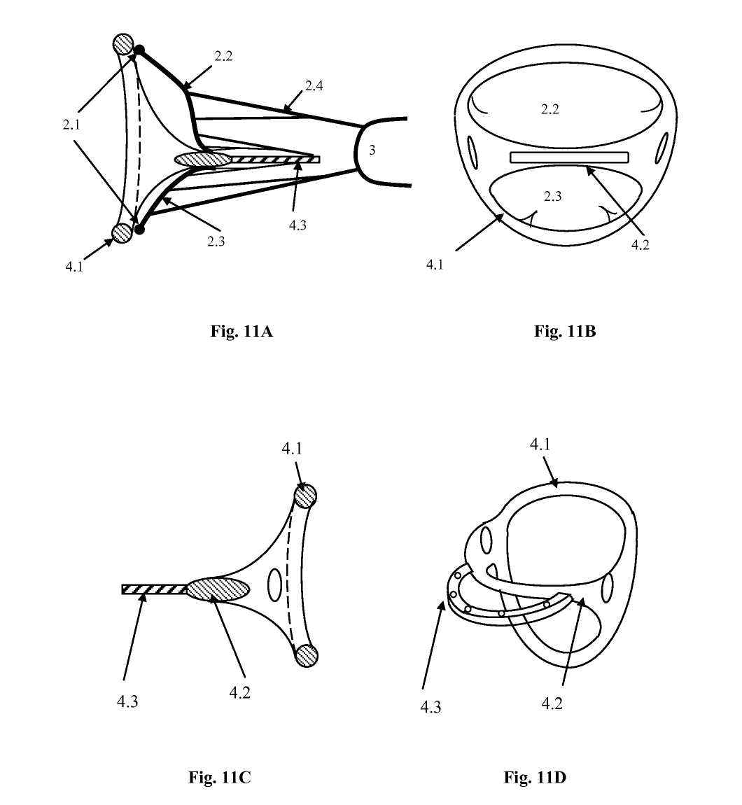

Reference is now made to FIGS. 11A-11D, wherein the coaptation plate is shown with an extension bar to help facilitate the attachment of chordae for translocation. The tip of tongue plate (4.2) can be extended with an extension bar with holes, which can be used to anchor artificial chordae (polytetrafluoroethylene) or other chordae repair. FIG. 11D shows the bar as part of the mitral valve coaptation plate. The holes in the lateral sides of the tongue plate (4.2) depend upon the commissural leaflet size and shape. The tongue plate can be a complete plate across two commissures or a partial plate with one commissure side.

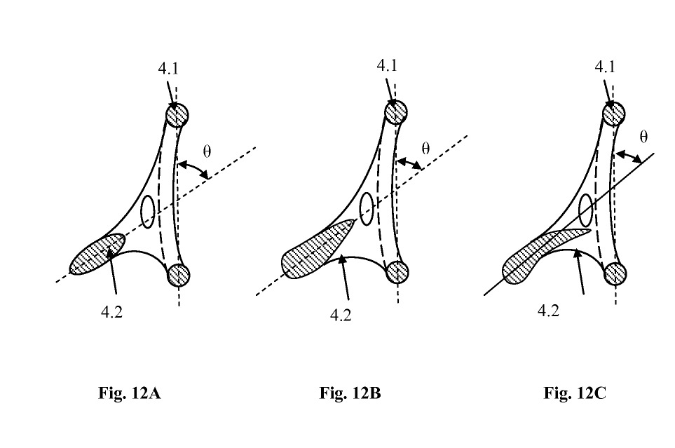

Reference is now made to FIGS. 12A-12C, wherein the mitral valve coaptation plate is shown with various plate shapes at an offset θ measured from the plane of the suture ring.

Artificial leaflets can be attached onto the suture ring to replace either or both mitral valve leaflets (2.2 or 2.3) to make an artificial heart valve. The leaflets are made of polyurethane or other materials. Artificial chordae are used to connect leaflets.

In brief, the mitral valve coaptation plate (4) as described herein supports leaflet coaptation substantially by providing fully forgiving matching of leaflet (2.2 and 2.3) position. The mitral valve coaptation plate is designed for different mechanisms of mitral valve regurgitation such as ischemic valve disease, mitral prolapse and chordae elongation or rupture.

Furthermore, the suture ring (4.1) restores a dilatated mitral valve annulus (2.1) in the septal-lateral diameter. This configuration along with the coaptation plate helps to reduce the gap between the leaflets.

The disclosed method and apparatus is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed method, except as may be outlined in the claims.

Alternative applications for this invention include using this apparatus and method as an alternative solution for a dysfunctional tricuspid valve, aortic valve, or pulmonary valve, in which a tripod coaptation plate is used. Consequently, any embodiments comprising a coaptation plate to support leaflets with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures and apparatus described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the apparatus and/or methods disclosed and claimed herein can be made and executed without undue experimentation in light of the present disclosure. While the apparatus and methods of this invention have been described in terms of preferred embodiments, it will be apparent to those skilled in the art that variations may be applied to the apparatus and/or methods and in the steps or in the sequence of steps of the method described herein without departing from the concept, spirit, and scope of the invention.

More specifically, it will be apparent that certain components which are both shape and material related may be substituted for the components described herein while the same or similar results would be achieved. All such similar substitutes and modifications apparent to those skilled in the art are deemed to be within the spirit, scope, and concept of the invention as defined by the appended claims.

### REFERENCES

1. Gelsomino S, Lorusso R, Caciolli S, et al., Insights on left ventricular and valvular mechanisms of recurrent ischemic mitral regurgitation after restrictive annuloplasty and coronary artery bypass grafting. J Thorac Cardiovasc Surg, 2008;136:507-18
2. Watanabe N, Ogasawara Y, Yamaura Y, et al., Quantitation of mitral valve tenting in ischemic mitral regurgitation by transthoracic real-time three-dimensional echocardiography. J Am Coll Cardiol, 2005;45:763-9
3. Bothe W, Nguyen T C, Ennis D B, et al., Effects of acute ischemic mitral regurgitation on three-dimensional mitral leaflet edge geometry. Eur J Cardiothorac Surg, 2008;33:191-7
4. Sakai T, Okita Y, Ueda Y, et al., Distance between mitral anulus and papillary muscles: anatomic study in normal human hearts. J Thorac Cardiovasc Surg, 1999;118:636-41
5. Sadeghpour A, Abtahi F, Kiavar M, et al., Echocardiographic evaluation of mitral geometry in functional mitral regurgitation. J Cardiothorac Surg, 2008;3:54
6. He S, Fontaine A A, Schwammenthal E, Yoganathan A P and Levine R A. Integrated mechanism for functional mitral regurgitation: leaflet restriction versus coapting force: in vitro studies. Circulation, 1997;96:1826-34
7. He S, Jimenez J, He Z and Yoganathan A P. Mitral leaflet geometry perturbations with papillary muscle displacement and annular dilatation: an in-vitro study of ischemic mitral regurgitation. J Heart Valve Dis, 2003;12:300-7
8. He S, Lemmon J D, Jr., Weston M W, et al., Mitral valve compensation for annular dilatation: in vitro study into the mechanisms of functional mitral regurgitation with an adjustable annulus model. J Heart Valve Dis, 1999;8:294-302
9. Levine R A, Hung J, Otsuji Y, et al., Mechanistic insights into functional mitral regurgitation. Curr Cardiol Rep, 2002;4:125-9

I claim:

1. An apparatus for repairing a regurgitating mitral heart valve, comprising: a suture ring to provide attachment to the mitral valve annulus and a tongue plate extending from said suture ring to a length no longer than the patient's mitral valve leaflet length to a position between the anterior and posterior leaflets at closure to match the free edges of the leaflets so as to block gaps between leaflets during systole.

2. The apparatus of claim 1, where said tongue plate extends from the commissural sides of said suture ring.

3. The apparatus of claim 1, further comprising a rod or rods extending from said tongue plate to secure chordae or artificial chordae.

4. The apparatus of claim 1, further comprising a bar extending from said tongue plate to secure chordae or artificial chordae.

5. The apparatus of claim 2, further comprising a rod or rods extending from said tongue plate to secure chordae or artificial chordae.

6. The apparatus of claim 2, further comprising a bar extending from said tongue plate to secure chordae or artificial chordae.

7. The apparatus of claim 3, further comprising an artificial leaflet (anterior or posterior) or leaflets (both anterior and posterior) attached to said suture ring with the leaflets chordae attached to said rod or rods.

8. The apparatus of claim 4, further comprising an artificial leaflet (anterior or posterior) or leaflets (both anterior and posterior) attached to said suture ring with the leaflets chordae attached to said bar.

9. The apparatus of claim 5, further comprising an artificial leaflet (anterior or posterior) or leaflets (both anterior and posterior) attached to said suture ring with the leaflets chordae attached to said rod or rods.

10. The apparatus of claim 6, further comprising an artificial leaflet (anterior or posterior) or leaflets (both anterior and posterior) attached to said suture ring with the leaflets chordae attached to said bar.

11. A method for repairing a regurgitating mitral heart valve, comprising the steps of: providing an apparatus comprising a suture ring to provide attachment to the mitral valve annulus and a tongue plate extending from the suture ring to a length no longer than the patient's mitral valve leaflet length to a position between the anterior and posterior leaflets at closure to match the free edges of the leaflets so as to block gaps between leaflets during systole; implanting said apparatus inside the patient's mitral valve.

12. The method of claim 11, where said apparatus provided has said tongue plate extending from the commissural sides of said suture ring.

13. The method of claim 11, further comprising the step of attaching an extension rod or rods to said tongue plate for chordal repair.

14. The method of claim 11, further comprising the step of attaching an extension bar to said tongue plate for chordal repair.

15. The method of claim 12, further comprising the step of attaching an extension rod or rods to said tongue plate for chordal repair.

16. The method of claim 12, further comprising the step of attaching an extension bar to said tongue plate for chordal repair.

17. The method of claim 13, further comprising the step of attaching an artificial leaflet (anterior or posterior) or leaflets (both anterior and posterior) to said suture ring with the leaflets chordae attached to said rod or rods.

18. The method of claim 14, further comprising the step of attaching an artificial leaflet (anterior or posterior) or leaflets (both anterior and posterior) to said suture ring with the leaflets chordae attached to said bar.

19. The method of claim 15, further comprising the step of attaching an artificial leaflet (anterior or posterior) or leaflets (both anterior and posterior) to said suture ring with the leaflets chordae attached to said rod or rods.

20. The method of claim 16, further comprising the step of attaching an artificial leaflet (anterior or posterior) or leaflets (both anterior and posterior) to said suture ring with the leaflets chordae attached to said bar.
